(19) United States
(12) Patent Application Publication
(10) Pub. No.: US 2011/0189770 A1
(43) Pub. Date: Aug. 4, 2011

(54) Endosome-Disrupting Compositions and Conjugates

(75) Inventor: Heather A. Clark, Lexington, MA (US)

(73) Assignee: The Charles Stark Draper Laboratory, Inc., Cambridge, MA (US)

(21) Appl. No.: 12/997,561

(22) PCT Filed: Jun. 12, 2009
(86) PCT No.: PCT/US09/03531

§ 371 Date: Apr. 22, 2011

Related U.S. Application Data
(60) Provisional application No. 61/131,786, filed on Jun. 12, 2008.

(51) Int. Cl. C12N 5/00; C08F 112/14

(52) U.S. Cl. 435/375; 525/333.6

(57) ABSTRACT

The present invention is directed to an endosome-disrupting conjugate, and methods of delivery of said conjugate to cells. The conjugates of the invention comprise a payload and an endosome-disrupting component of which the latter comprises a plurality of moieties that react under acidic conditions, e.g., within an endosome, to release gaseous endosome-disrupting molecules, e.g., CO₂ and/or O₂. The payload portion of the conjugate comprises molecules or particles that perform a function, e.g., diagnostic or therapeutic, within cells. The endosome-disrupting component may be selected from polymers or polymeric capsules comprising the endosome-disrupting moieties of the invention. The conjugates optionally further comprise targeting moieties.

### RELATED APPLICATION

This application claims the benefit of U.S. Provisional Application No. 61/131,786, filed Jun. 12, 2008, the contents of which are incorporated herein by reference in their entirety.

### BACKGROUND OF THE INVENTION

In the evolving field of drug discovery and molecular and pharmaceutical biology, a need exists for effective mechanisms for delivering therapeutic agents to functional components of cells. One obstacle to accomplishing this goal is encapsulation of the therapeutic agent in an endosomal vesicle within a cell. The endosome sequesters the therapeutic agent from an intended target within the cytosol of the cell and additionally subjects the therapeutic agent to the low pH and active degradatory machinery of the endosome/lysosome.

Various reagents (e.g., chloroquine, polyethyleneimine (PEI), certain highly charged cationic compounds, fusogenic peptides, and inactivated adenoviruses) have been developed that are intended to quickly disrupt the endosome in order to minimize the amount of time that a therapeutic agent spends in this hostile environment; however, there exist drawbacks to the use of these agents. For example, alkaline endosomolytic agents such as chloroquine and polyethyleneimine function by neutralizing the intra-endosomal pH, decreasing the activity of the lysosomal enzymes. However, the high local concentrations of these agents required to effect lysis of an endosome are potentially cytotoxic and may evoke cellular immune responses. Therefore, alternative techniques for delivering therapeutic agents are needed.

### SUMMARY OF THE INVENTION

The present invention is directed to an endosome-disrupting conjugate, and methods of delivery of said conjugate to cells. The conjugates of the invention comprise a payload and an endosome-disrupting component of which the latter comprises a plurality of moieties that react under acidic conditions, e.g., within an endosome, to release gaseous endosome-disrupting molecules, e.g. CO₂ and/or O₂. The payload portion of the conjugate comprises molecules or particles that perform a function, e.g., diagnostic or therapeutic, within cells. In certain embodiments, the endosome-disrupting component is selected from polymers or polymeric capsules comprising the endosome-disrupting moieties of the invention. The components of the conjugate, i.e., the endosome-disrupting component, the moieties, and the payload, may be associated through any combination of covalent or non-covalent bonds or the encapsulation of one or more components. The conjugates optionally further comprise targeting moieties.

### DESCRIPTION OF FIGURES

FIG. 1. A cell delivery system for delivering the conjugate 1 of the invention, wherein a conjugate 1 bearing a payload 2 is shuttled into a cell through endocytosis 5 and endosome-disrupting components generate gaseous endosome-disrupting molecules 8 allowing the conjugate to escape from the endosome.

FIG. 2. Schematically depicts conjugates comprising endosome-disrupting moieties 15: a. enclosed within swellable and/or biodegradable polymer capsules 14 bound through a linker 13 to the payload 12, b. enclosed within multiple capsules 14 bound through linkers 13 to the payload 12, c. enclosed within multiple capsules 14 bound through bridged linkers 13 to the payload 12, d. wherein a payload 12 is encapsulated within a swellable and/or biodegradable capsule 16 further comprising endosome-disrupting moieties, and e. enclosed within polymeric capsules 14 encapsulated with the payload 12 within a swellable and/or biodegradable polymer 16.

FIG. 3. Schematically depicts conjugates wherein: a. a polymer comprising endosome-disrupting moieties 17 is bound to a payload 12, b. multiple polymeric chains 17 are bound to a payload 12, c. polymeric chains 17 branch from a payload 12, d. the payload 12 is encapsulated within polymer 17, and e. polymer and payload are encapsulated within a swellable and/or biodegradable polymer 16.

FIG. 4. Schematic of an exemplary process for modifying the amine functionality on a polystyrene bead to form a conjugate followed by reaction under acidic conditions to release the endosome-disrupting moieties.

FIG. 5. Experimental design of the in vitro testing of the cellular delivery system. Each circle represented two culture dishes, i.e., each test was done in duplicate, where the far left dish contains HEK cells with unmodified amine beads, the second dish from the left contains HEK cells with carbonate-modified beads, the central dish contains HEK cells and FM 1-43 Dye, the forth dish from the left contains HEK cells with unmodified amine beads and FM 1-43 dye, and finally the rightmost dish, i.e., the experimental group, contains HEK cells comprising carbonate-modified beads and FM 1-43 dye.

FIG. 6. Image of HEK cell with unmodified amine beads after overnight incubation on a 63× oil objective at the wavelengths (excitation/emission) 488/505-545 nm for the polystyrene beads and 488/650-710 nm for FM 1-43.

FIG. 7. Image of a HEK cell with carbonate modified beads after overnight incubation on a 63× oil objective at the wavelengths (excitation/emission) 488/505-545 nm for the polystyrene beads and 488/650-710 nm for FM 1-43.

### DETAILED DESCRIPTION OF THE INVENTION

The present invention relates to compositions and methods for delivering a molecule or particle of interest, i.e., a payload, into eukaryotic cells, particularly into the cytoplasm, in vitro or in vivo. In one aspect, the present invention provides a conjugate comprising an endosome-disrupting component and a payload. The endosome-disrupting component of the invention is capable of effecting the lysis or degradation of an endosomal membrane by releasing gaseous endosome-disrupting molecules in response to a change in pH. The payload of the conjugate is selected from particles, such as diagnostic sensors, or molecules, such as therapeutics, intended for delivery to the cytosol of a cell. The conjugates may further comprise components which allow for the targeting of conjugates to cells or components of cells and groups that improve the biocompatibility of the conjugates.

The term endosome as used herein refers to an intracellular vesicle involved in cellular digestion. Endosome as used herein is not limited to one particular type of intracellular vesicle or stage of intracellular digestion. Endosome includes, for example, early endosomes, late endosomes and lysosomes.

In certain embodiments, the invention provides a method for delivering a conjugate 1 comprising a payload 2 into the cytosol 11 of a cell (FIG. 1). In certain exemplary embodiments, the conjugate 1 contacts cellular receptors 5 and, for example, is endocytotically delivered to the interior of a cell 5. The conjugate 1 becomes encapsulated in an endosomal vesicle 6 within the cytosol 11 and as the pH of the endosome decreases, the conjugate releases gaseous endosome-disrupting molecules 8. The gaseous endosome-disrupting molecules 8 effect the degradation or rupturing of the endosomal membrane 9. The chemically-modified conjugate 10 is then freed from the endosome into the cytosol 11 of the cell, delivering the payload 2 to a desired location within the cytosol.

In certain embodiments, the endosome-disrupting component is selected from polymers, polymeric capsules, dendrimers or a combination thereof. In certain aspects, the endosome-disrupting component of the invention comprises moieties that react to release gaseous endosome-disrupting molecules under acidic conditions.

Gaseous endosome-disrupting molecules of the invention include molecules that, in their pure form, exist as a gas under standard temperature and pressure, i.e., 273.15° Kelvin and 760 torr. Exemplary endosome-disrupting molecules include carbon dioxide and oxygen. In certain embodiments, at the time of their release, the endosome-disrupting molecules are in a gaseous state, while in other embodiments, they may dissolve in the surrounding medium, such as the endosomal contents. Of course, following release from the conjugates, endosome-disrupting molecules may exist in an equilibrium between gaseous and solubilized states.

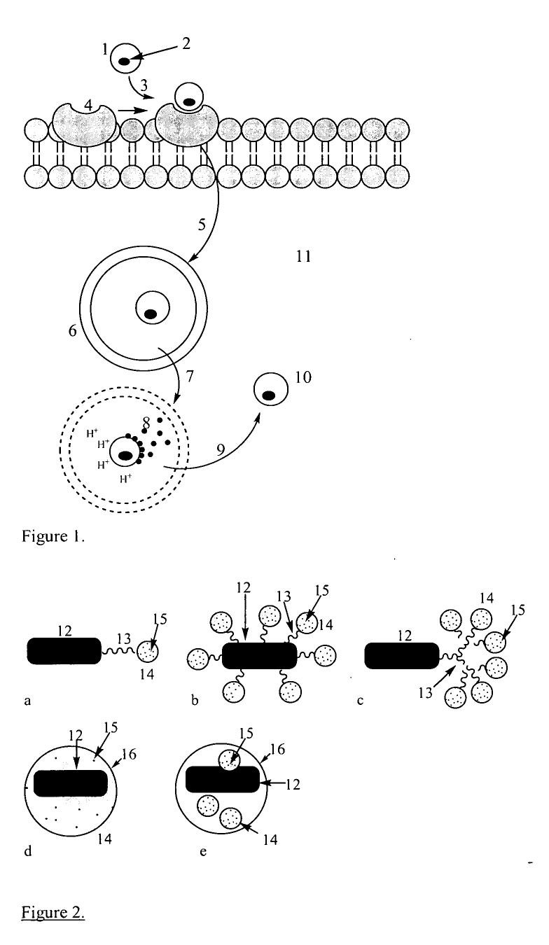

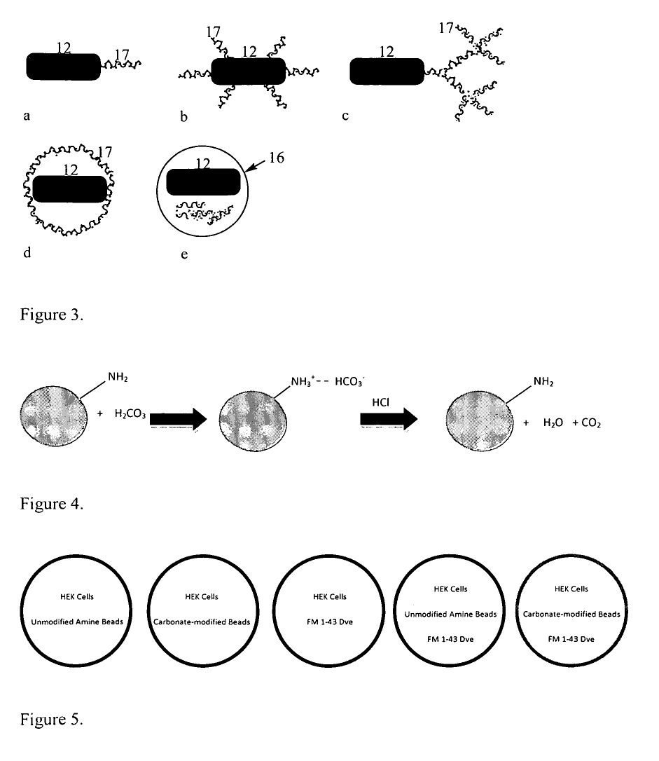

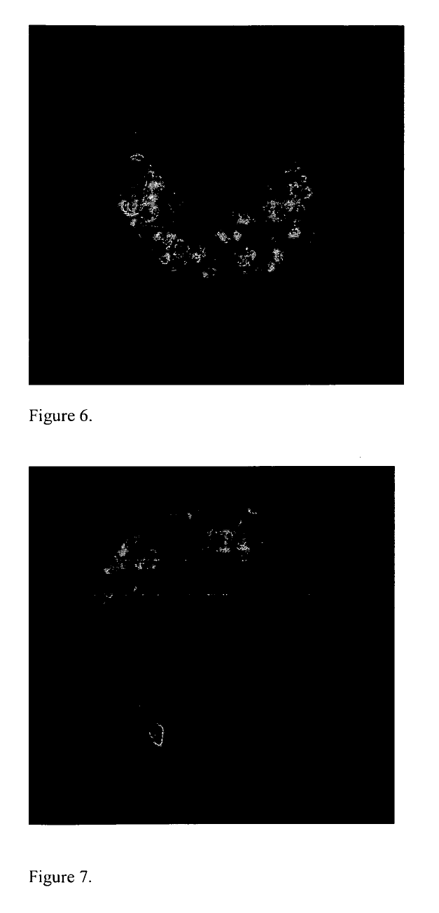

In certain embodiments, the endosome-disrupting component is a polymeric capsule comprising one or more endosome-disrupting moieties, i.e. molecules that react to afford endosome-disrupting molecules, e.g., ionic carbonates, metal carbonates or peroxides (FIG. 2). In certain embodiments, the encapsulating polymer is a biodegradable and/or swellable polymer that reacts with acid. In certain embodiments, the encapsulating polymer of the capsule degrades and/or swells under physiologic acidic conditions. In certain embodiments, the endosome-disrupting component is a polymer comprising gas-releasing moieties, e.g., carbonates or peroxides, that react to release gaseous endosome-disrupting molecules under acidic conditions (FIG. 3). In an exemplary embodiment wherein the gaseous molecules are released from a polymer of the conjugate, a carbonate moiety of the polymer hydrolyzes in acidic physiologic medium affording carbon dioxide. The released carbon dioxide molecules may initially exist in a solubilized state until a critical concentration is reached, upon which bubbles or other gaseous pockets may begin to form.

In certain aspects, the endosome-disrupting component is a dendrimer comprising gas-releasing moieties, e.g., carbonates or peroxides, that react to release gaseous endosome-disrupting molecules under acidic conditions. The gas-releasing moieties of the dendrimer may be incorporated into the scaffold of the dendrimer, i.e., chemically bound to moieties in the dendrimer scaffold, or associated with the dendrimer through intermolecular forces, e.g., hydrogen bonding or ionic bonding between dendrimer moieties and gas-releasing moieties. In certain embodiments, the dendrimers may have hydrophilic surface functionalities to allow for enhanced solubility of the dendrimers, e.g., carboxyl groups or ammonium groups. The dendrimers may have hydrophobic interiors, comprising, e.g., alkyl or phenyl groups, suitable for transporting hydrophobic drug molecules. In other embodiments, the dendrimers may have hydrophilic interiors capable of transporting hydrophilic drugs and/or hydrophilic gas-releasing moieties, e.g., peroxides or carbonates. In certain embodiments, the dendrimer is a polymer.

In certain embodiments, the acidic conditions under which the polymers of the invention swell and/or degrade comprise a pH from 4-7. In certain embodiments, the acidic conditions comprise a pH from 5-6. In certain embodiments, the gaseous endosome-disrupting molecules released from the endosome-disrupting component comprises O₂ and/or CO₂. In certain embodiments, the moieties of the endosome-disrupting component comprise carbonates, polycarbonates or peroxides.

In certain embodiments, the conjugates comprise endosome-disrupting moieties 15 incorporated within swellable and/or biodegradable polymeric capsules 14 (FIG. 2). In certain embodiments, the conjugates comprise one or more metal carbonate particles 15 incorporated within swellable and/or biodegradable polymeric capsules 14. In certain embodiments, the metal carbonate particles 15 within the capsules 14 are selected from sodium carbonate, sodium bicarbonate, calcium carbonate, potassium carbonate, potassium bicarbonate, lithium carbonate, cesium carbonate or any combination thereof. In certain such embodiments, one or more metal carbonate particle 15 are protected from the biological medium within the polymer until reaching an acidic environment, such as an endosome, wherein the polymer degrades and/or swells to release the metal carbonates and/or expose them to the surrounding medium. Under acidic conditions, metal carbonates rapidly react, releasing carbon dioxide. In certain embodiments, the polymer surrounding the metal carbonate comprises gas-releasing functionalities of the invention, such as a polycarbonate polymer.

In certain embodiments, one or more of the moieties are ionic. In certain embodiments, the ionic moieties comprise carbonate ions. In particular embodiments, the carbonate ions are associated with one or more components of the conjugate through ionic interactions. In certain embodiments, the endosome-disrupting component comprises carbonate ions.

The term ion as used herein refers to an atom or molecule that has a net positive or net negative electrical charge, e.g., Na⁺, Ca⁺⁺, Cl⁻, SO₄⁻, etc., as is well understood in the art.

In certain embodiments, one or more of the gaseous endosome-disrupting moieties 15 within the capsules 14 are peroxides. In certain embodiments, the peroxides comprise hydroperoxides, organic peroxides or peroxyacids. In certain such embodiments, peroxide molecules are protected from the biological medium within the polymer until reaching an acidic environment, such as an endosome, wherein the polymer degrades and/or swells to release the peroxide molecules. The peroxides react with intracellular enzymes, e.g., catalase, to release oxygen. In certain embodiments, the polymer surrounding the peroxide comprises gas-releasing moieties, such as a polycarbonates or peroxides.

In certain embodiments, the polymer of the endosome-disrupting component is selected from a linear, branched (e.g., dendrimers), or cross-linked polymer or a combination thereof. In some embodiments, the backbone of the polymer comprises moieties, e.g., carbonate or peroxide functional groups, that react under acidic pH to release gaseous molecules. In other embodiments, the branches of a polymer comprise moieties that react to release gas. In certain embodiments, the backbone of the polymer and the branches comprise moieties that react to release gas. In certain embodiments, hydrolysable functionalities that do not release gas, such as esters or acetals, form part of the backbone or branches of the polymer.

In some embodiments, the polymers comprise carbonate functionalities as gas-releasing moieties. In certain embodiments, the polymers with carbonate functionalities are composed of alkylene carbonate moieties wherein the alkylene carbons are optionally substituted by heteroatom-containing functional groups such as hydroxyls, amines, acyl, acyloxy or alkoxy groups. Polyesters of carbonic acid are known to be hydrolysable under physiological pH, e.g., in the presence of hydrolytic enzymes, e.g., esterases; however, particular substituents on the polycarbonate can be selectively chosen (e.g., based on steric or electronic factors) to control the rate of hydrolysis in vitro or in vivo. For example, the rate of hydrolysis can be modified by altering the chain length and substitution pattern of alkylene carbonic acid units, as in International Application No. 93/20126, the disclosure of which is incorporated herein in its entirety by reference.

In certain embodiments, the polymers comprise peroxide functionalities as gas-releasing moieties. In certain embodiments, the polymers with peroxide functionalities are composed of organic peroxides, hydroperoxides or peroxy acids. Enzymes such as catalase are known to break down cytotoxic hydrogen peroxide into oxygen and water. In certain embodiments, peroxides of the conjugates react with enzymes to release O₂ gas.

In certain embodiments, the conjugate of the invention comprises a payload covalently bound to one or more endosome-disrupting components. The payload and endosome-disrupting component of the invention may be covalently bound in one or more locations on the payload or the endosome-disrupting component as depicted in exemplary configurations in FIGS. 2 and 3. In certain embodiments, the endosome-disrupting component is one or more groups, such as polymers 17 in FIG. 3 or swellable and/or biodegradable polymeric capsules 14 encapsulating metal carbonates or peroxides 15 as in FIG. 2. In certain embodiments, an endosome-disrupting component 14/17 is bound to one location of a payload 12 as in FIGS. 2a and 3a. In certain embodiments, multiple endosome-disrupting components 14/17 are bound to a payload 12 as in FIGS. 2b and 3b. In certain embodiments, multiple endosome-disrupting components 14/17 are bound to the payload 12 through branched linkages 13. In other embodiments, the payload 12 is contained within a swellable and/or biodegradable polymer 16 which also encapsulates the endosome-disrupting component 14/17 such as in FIGS. 2d, 2e or 3e. In certain embodiments, the endosome-disrupting component is a polymer 17 which encapsulates the payload 12 as seen in FIG. 3d. In certain embodiments, the conjugates may further comprise linkers 13 to bind the polymers 17 or polymeric capsules 14 to the payload 12. In exemplary embodiments, the endosome-disrupting component 14/17 and payload 12 are bound in any combination of the configurations depicted in FIGS. 2 and 3. For example, a payload 12 may be bound to multiple endosome-disrupting components 14/17 through branched linkages, combining components of FIGS. 2b and 2c or 3b and 3c. In other exemplary embodiments, the endosome-disrupting component 14/17 is bound to the payload 12 and encapsulated within a biodegradable and/or swellable polymer 16, combining, for example, FIGS. 2a and 2e and FIGS. 3a and 3d.

Not wishing to be bound by any one particular representation, the conjugates of the present invention may result from any number of chemical interactions. As would be clear to one of skill in the art, there are a variety of ways to associate one or more molecules into the conjugates of the invention through chemical bonding or encapsulation. In certain embodiments, one or more components or any portion of a component of the conjugates are covalently or non-covalently bound to the remainder of the conjugate. For example, a component such as the endosome-disrupting component may be associated with the payload through a covalent or non-covalent bond or in the event of more than one bond between the endosome-disrupting component and the payload, there may be any combination of covalent and non-covalent bonds such as all covalent bonds, all non-covalent bonds, or a mixture of covalent and non-covalent bonds. In certain embodiments, a moiety may be associated through any of covalent bonds, non-covalent bonds or a combination thereof. In certain embodiments, one or more of the moieties are bound to the endosome-disrupting component, or any other component of the conjugate, through one or more covalent or non-covalent bonds. For example, one moiety may be non-covalently associated, e.g., through an ionic or hydrogen bond, with the endosome-disrupting component while another moiety is covalently bound to the endosome-disrupting component. In particular embodiments, one or more of the moieties are carbonate ions. In certain embodiments, carbonate ions are associated with any portion of the conjugate such as the payload and/or the endosome disrupting-component.

The term covalent in reference to chemical bonding as used herein is characterized by the sharing of pairs of electrons between atoms, or between atoms and other covalent bonds. Covalent bonding includes σ-bonding, π-bonding, metal to non-metal bonding, agostic interactions, and three-center two-electron bonds.

The term non-covalent in reference to chemical bonding as used herein is characterized by a bond that does not involve the sharing of pairs of electrons, but rather involves more dispersed variations of electromagnetic interactions. Non-covalent interactions include hydrogen bonds, ionic bonds, Van der Waals forces, and hydrophobic interactions.

In certain aspects, the polymers or polymeric capsules of the conjugates are purchased or prepared synthetically to form the conjugates of the invention. In certain embodiments, the polymers or polymeric capsules comprise polycarbonates. In certain embodiments, the polymers or polymeric capsules comprise dendrimers. In certain embodiments, suitable polycarbonates can be prepared by known methods, e.g., by reacting: a diol with phosgene as in French Patent No. 905,141, U.S. Pat. No. 2,999,844, and German Patent Nos. 117,625; and 118,536,7; a diol with a bis(chloroformate) as in German Patent No. 857,948; a diol with a dialkyl carbonate as in Carothers, W. H. et al. J. Am. Chem. Soc., 1930, 314, 52; Hill, J. H. et al. J. Am. Chem., Soc., 1933, 5031, 55; and Sarel, S. et al. J. Org. Chem., 1959, 1873, 24; a diol with urea as in European Patent No. 0,057,825; as well as by polycondensation of bis(alkyl carbonates) as in U.S. Pat. No. 2,789,968; ring opening polymerization of cyclic carbonates or ring opening polymerization of spiroortho carbonates as in Sakai S. et al. J. Polym. Sci., Polym. Lett. Ed. 1973, 631, 11 and Endo, T. et al. J. Polym. Sci, Polym. Chem. Ed. 1975, 2525, 13; and by copolymerization of epoxides as in Inoue, S. et al. J. Polym. Sci. B, 1969, 287; and U.S. Pat. Nos. 3,900,424, 3,953,383, and 4,665,136. Other exemplary polycarbonates that can be used as gas-releasing endosomolytic agents of the invention and methods of their preparation are disclosed in U.S. Pat. Nos. 3,301,824, 4,243,775, 4,429,080, 4,857,602, 4,882,168, 5,066,772, 5,366,756, 5,403,347, 5,522,841 and EP Patent No. 0390860.

In certain embodiments, the polycarbonate is synthesized by reacting a carbonic acid derivative (such as phosgene, carbonyl diimidazole, tetramethyl orthocarbonate, etc.) with an alcohol, wherein the alcohol is a polyol such as a reactive sugar alcohol or glycerol having one or more protected secondary hydroxyl groups and two free primary hydroxyl groups. A wide array of strategies and techniques for selective protection of polyols such as sugar alcohols are known, such as, e.g., the pre-protection of the primary terminal hydroxyl groups, e.g., by converting them into benzoic acid ester groups, converting secondary hydroxyl groups to acetals or hemiacetals with, for example, acetone (giving rise to O-isopropylidene residues) and cleaving benzoic acid ester groups with, for example, methanol in the presence of sodium methoxide. The partially protected sugar alcohols have two free terminal primary hydroxyl groups and protected secondary hydroxyl groups and can be used as a diol for the production of the polycarbonates.

For functionalization with carbonic acid ester residues, polycarbonates or other polymers having free hydroxyl or amine groups can be reacted with, for example, active carbonic acid derivatives, e.g., chloroformic acid esters or dicarbonates. Carbamates of hydroxy compounds are generally made, e.g., by their conversion with isocyanates or with carbamoyl chlorides. Alternately derivatives of amine compounds can be formed, e.g., by reaction of amines with chloroformate, isocyanates or acid derivatives. The residues may also comprise ortho ester residues, e.g., those of an ortho carboxylic acid ester or an ortho carbonic acid ester, which are acid sensitive and thus increase the biodegradability of the polyesters of the invention.

Hemi-acetal or acetal groups may be removed by methods such as stirring in water and trifluoroacetic acid, which provides polycarbonates having free secondary hydroxyl groups. The deprotected secondary hydroxyl groups can be derivatized as described in Harrison, “Compendium of Organic Synthetic Methods”, Vols. I-IV, 1971-1980 and Wilkinson, S. G. “Comprehensive Organic Chemistry”, D. Barton and W. D. Ollis, Eds., Vol. I, p. 579, 1979. For derivatization to carboxylic ester residues, the polyesters having free hydroxylic groups are preferably dissolved or suspended in an inert, aprotic solvent such as tetrahydrofuran, methylene chloride, toluene, or dimethylformamide and reacted in the presence of a catalyst, such as a tertiary amine, with an active carboxylic acid derivative. Active carboxylic acid derivatives are, for example, carboxylic acid anhydrides and carboxylic acid chlorides. These derivatives may be obtained by reacting the carboxylic acid with an activation reagent and can often be brought into contact with the hydroxyl groups when formed in situ. Reaction with ketenes leads also to the introduction of carboxylic acid ester residues.

In certain aspects, exemplary swellable polymers of U.S. Pat. Nos. 3,997,484, 3,669,103 and 3,670,731 are included herein by reference. Representative biodegradable polymers include herein comprise poly[lactide-co-glycolide], polyanhydrides, and polyorthoesters, polyanhydrides and polyesters.

In certain embodiments, the conjugate further comprises a targeting moiety. In certain embodiments, the targeting moiety is bound to a portion of the conjugate such as the endosome-disrupting component or the payload. The targeting moiety, which assists the sensor in localizing to a particular target area, entering a target cell(s), and/or locating proximal to an ion channel, may be selected on the basis of the particular condition or site to be monitored. The targeting moiety may further comprise any of a number of different chemical entities. In one embodiment, the targeting moiety is a small molecule. Molecules which may be suitable for use as targeting moieties in the present invention include haptens, epitopes, and dsDNA fragments and analogs and derivatives thereof. Such moieties bind specifically to antibodies, fragments or analogs thereof, including mimetics (for haptens and epitopes), and zinc finger proteins (for dsDNA fragments). Nutrients believed to trigger receptor-mediated endocytosis and therefore useful targeting moieties include biotin, folate, riboflavin, carnitine, inositol, lipoic acid, niacin, pantothenic acid, thiamin, pyridoxal, ascorbic acid, and the lipid soluble vitamins A, D, E and K. Another exemplary type of small molecule targeting moiety includes steroidal lipids, such as cholesterol, and steroidal hormones, such as estradiol, testosterone, etc.

In another embodiment, the targeting moiety may comprise a protein. Particular types of proteins may be selected based on known characteristics of the target site or target cells. For example, the probe can be an antibody either monoclonal or polyclonal, where a corresponding antigen is displayed at the target site. In situations wherein a certain receptor is expressed by the target cells, the targeting moiety may comprise a protein or peptidomimetic ligand capable of binding to that receptor. Protein ligands of known cell surface receptors include low density lipoproteins, transferrin, insulin, fibrinolytic enzymes, anti-HER2, platelet binding proteins such as annexins, and biological response modifiers (including interleukin, interferon, erythropoietin and colony-stimulating factor). A number of monoclonal antibodies that bind to a specific type of cell have been developed, including monoclonal antibodies specific for tumor-associated antigens in humans. Among the many such monoclonal antibodies that may be used are anti-TAC, or other interleukin-2 receptor antibodies; 9.2.27 and NR-ML-05 to the 250 kilodalton human melanoma-associated proteoglycan; and NR-LU-10 to a pancarcinoma glycoprotein. An antibody employed in the present invention may be an intact (whole) molecule, a fragment thereof, or a functional equivalent thereof. Examples of antibody fragments are F(ab′)₂, Fab′, Fab, and F, fragments, which may be produced by conventional methods or by genetic or protein engineering.

Other preferred targeting moieties include sugars (e.g., glucose, fucose, galactose, mannose) that are recognized by target-specific receptors. For example, instant claimed constructs can be glycosylated with mannose residues (e.g., attached as C-glycosides to a free nitrogen) to yield targeted constructs having higher affinity binding to tumors expressing mannose receptors (e.g., glioblastomas and gangliocytomas), and bacteria, which are also known to express mannose receptors (Bertozzi, C R and M D Bednarski Carbohydrate Research 223:243 (1992); J. Am. Chem. Soc. 114:2242,5543 (1992)), as well as potentially other infectious agents. Certain cells, such as malignant cells and blood cells (e.g., A, AB, B, etc.) display particular carbohydrates, for which a corresponding lectin may serve as a targeting moiety.

In certain aspects, any payload molecule or particle having biological activity, therapeutic or diagnostic utility may be delivered to cells using the conjugates of the invention. Exemplary payloads of the invention include proteins, polypeptides, polynucleotides, nucleoproteins, polysaccharides, glycoproteins, lipoproteins, and synthetic and biologically engineered analogs thereof.

Examples of biologically active compounds that might be utilized in a delivery application of the invention include literally any hydrophilic or hydrophobic biologically active compound. Preferably, though not necessarily, the drug is one that has already been deemed safe and effective for use by the appropriate governmental agency or body. For example, drugs for human use listed by the FDA under 21 C.F.R. sections 330.5, 331 through sections 361; 440-460; drugs for veterinary use listed by the FDA under 21 C.F.R. sections 500-582, incorporated herein by reference, are all considered acceptable for use in the present inventive cell delivery composition.

Biologically active compounds for use in the present invention include any pharmacologically active substances that produce a local or systemic effect in animals, preferably mammals, or humans. The term thus means any substance intended for use in the diagnosis, cure, mitigation, treatment or prevention of disease or in the enhancement of desirable physical or mental development and conditions in an animal or human.

Classes of pharmaceutically active compounds that can be used in the practice of the present invention include, but are not limited to, anti-AIDS substances, anti-cancer substances, antibiotics, immunosuppressants (e.g., cyclosporine), anti-viral substances, enzyme inhibitors, neurotoxins, opioids, hypnotics, antihistamines, lubricants tranquilizers, anti-convulsants, muscle relaxants and anti-Parkinson substances, anti-spasmodics and muscle contractants, miotics and anti-cholinergics, anti-glaucoma compounds, anti-parasite and/or anti-protozoal compounds, anti-hypertensives, analgesics, anti-pyretics and anti-inflammatory agents such as NSAIDs, local anesthetics, ophthalmics, prostaglandins, anti-depressants, anti-psychotic substances, anti-emetics, imaging agents, specific targeting agents, neurotransmitters, proteins, cell response modifiers, vaccines, ribozymes, anti-sense agents, and RNA.

A more complete listing of classes of compounds suitable for delivery into cells according to the present invention may be found in the Pharmazeutische Wirkstoffe (Von Kleemann et al. (eds) Stuttgart/N.Y., 1987, incorporated herein by reference). Examples of particular pharmaceutically active substances are presented below:

Anti-AIDS substances are substances used to treat or prevent Autoimmune Deficiency Syndrome (AIDS). Examples of such substances include CD4,3′-azido-3′-deoxythymidine (AZT), 9-(2-hydroxyethoxymethyl)-guanine (acyclovir), phosphonoformic acid, 1-adamantanamine, peptide T, and 2′,3′ dideoxycytidine.

Anti-cancer substances are substances used to treat or prevent cancer. Examples of such substances include methotrexate, cisplatin, prednisone, hydroxyprogesterone, medroxyprogesterone acetate, megestrol acetate, diethylstilbestrol, testosterone propionate, fluoxymesterone, vinblastine, vincristine, vindesine, daunorubicin, doxorubicin, hydroxyurea, procarbazine, aminoglutethimide, mechlorethamine, cyclophosphamide, melphalan, uracil mustard, chlorambucil, busulfan, carmustine, lomusline, dacarbazine (DTIC: dimethyltriazenomidazolecarboxamide), methotrexate, fluorouracil, 5-fluorouracil, cytarabine, cytosine arabinoxide, mercaptopurine, 6-mercaptopurine, and thioguanine.

Antibiotics are art recognized and are substances which inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromycin and cephalosporins.

Anti-viral agents are substances capable of destroying or suppressing the replication of viruses. Examples of anti-viral agents include a-methyl-P-adamantane methylamine, 1,-D-ribofuranosyl-1,2,4-triazole-3 carboxamide, 9-[2-hydroxy-ethoxy]methylguanine, adamantanamine, 5-iodo-2′-deoxyuridine, trifluorothymidine, interferon, and adenine arabinoside.

Enzyme inhibitors are substances which inhibit an enzymatic reaction. Examples of enzyme inhibitors include edrophonium chloride, N-methylphysostigmine, neostigmine bromide, physostigmine sulfate, tacrine HCl, tacrine, 1-hydroxy maleate, iodotubercidin, p-bromotetramisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N6-monomethyl-L-arginine acetate, carbidopa, 3-hydroxybenzylhydrazine HCl, hydralazine HCl, clorgyline HCl, deprenyl HCl, L(−)-, deprenyl HCl, D(+)-, hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-a-methylbenzyl amine (DCMB), 8,9-dichloro-2,3,4,5-tetrahydro-1H-2-benzazepine hydrochloride, p-aminoglutethimide, p-aminoglutethimide tartrate, R(+)-p-aminoglutethimide tartrate, S(−)-3-iodotyrosine, alpha-methyltyrosine, L-, alpha-methyltyrosine, D L-acetazolamide, dichlorphenamide, 6-hydroxy-2-benzothiazolesulfenamide, and allopurinol.

Neurotoxins are substances which have a toxic effect on the nervous system, e.g., nerve cells. Neurotoxins include adrenergic neurotoxins, cholinergic neurotoxins, dopaminergic neurotoxins, and other neurotoxins. Examples of adrenergic neurotoxins include N-(2-chloroethyl)-N-ethyl-2-bromobenzylamine hydrochloride. Examples of cholinergic neurotoxins include acetylethylcholine mustard hydrochloride. Examples of dopaminergic neurotoxins include 6-hydroxydopamine HBr, 1-methyl-4-(2-methylphenyl)-1,2,3,6-tetrahydro-pyridine hydrochloride, 1-methyl-4-phenyl-2,3-dihydropyridinium perchlorate, N-methyl-4-phenyl-1,2,5,6 tetrahydropyridine HCl, and 1-methyl-4-phenylpyridinium iodide.

Opioids are substances having opiate like effects that are not derived from opium. Opioids include opioid agonists and opioid antagonists. Opioid agonists include codeine sulfate, fentanyl citrate, hydrocodone bitartrate, loperamide HCl, morphine sulfate, noscapine, norcodeine, normorphine, and thebaine. Opioid antagonists include nor-binaltorphimine HCl, buprenorphine, chlornaltrexamine 2HCl, funaltrexamione HCl, nalbuphine HCl, nalorphine HCl, naloxone HCl, naloxonazine, naltrexone HCl, and naltrindole HCl.

Hypnotics are substances which produce a hypnotic effect. Hypnotics include pentobarbital sodium, phenobarbital, secobarbital, thiopental and mixtures thereof, heterocyclic hypnotics, dioxopiperidines, glutarimides, diethyl isovaleramide, a-bromoisovaleryl urea, urethanes and disulfanes.

Antihistamines are substances which competitively inhibit the effects of histamines. Examples include pyrilamine, chlorpheniramine, tetrahydrazoline, and the like.

Lubricants are substances that increase the lubricity of the environment into which they are delivered. Examples of biologically active lubricants include water and saline.

Tranquilizers are substances which provide a tranquilizing effect. Examples of tranquilizers include chloropromazine, promazine, fluphenazine, reserpine, deserpidine, and meprobamate.

Anti-convulsants are substances which have an effect of preventing, reducing, or eliminating convulsions. Examples of such agents include primidone, phenyloin, valproate, Chk and ethosuximide.

Muscle relaxants and anti-Parkinson agents are agents which relax muscles or reduce or eliminate symptoms associated with Parkinson's disease. Examples of such agents include mephenesin, methocarbomal, cyclobenzaprine hydrochloride, trihexyphenidyl hydrochloride, levodopa/carbidopa, and biperiden.

Anti-spasmodics and muscle contractants are substances capable of preventing or relieving muscle spasms or contractions. Examples of such agents include atropine, scopolamine, oxyphenonium, and papaverine.

Miotics and anti-cholinergics are compounds which cause bronchodilation. Examples include echothiophate, pilocarpine, physostigmine salicylate, diisopropylfluorophosphate, epinephrine, neostigmine, carbachol, methacholine, bethanechol, and the like.

Anti-glaucoma compounds include betaxolol, pilocarpine, timolol, timolol salts, and combinations of timolol, and/or its salts, with pilocarpine.

Anti-parasitics, -protozoals and -fungals include ivermectin, pyrimethamine, trisulfapyrimidine, clindamycin, amphotericin B, nystatin, flucytosine, natamycin, and miconazole.

Anti-hypertensives are substances capable of counteracting high blood pressure. Examples of such substances include alpha-methyldopa and the pivaloyloxyethyl ester of alpha-methyldopa.

Analgesics are substances capable of preventing, reducing, or relieving pain. Examples of analgesics include morphine sulfate, codeine sulfate, meperidine, and nalorphine.

Anti-pyretics are substances capable of relieving or reducing fever and anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Local anesthetics are substances which have an anesthetic effect in a localized region. Examples of such anesthetics include procaine, lidocaine, tetracaine and dibucaine.

Ophthalmics include diagnostic agents such as sodium fluorescein, rose bengal, methacholine, adrenaline, cocaine, and atropine. Ophthalmic surgical additives include alpha-chymotrypsin and hyaluronidase.

Prostaglandins are art recognized and are a class of naturally occurring chemically related, long-chain hydroxy fatty acids that have a variety of biological effects.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, pheneizine, and isocarboxazide.

Anti-psychotic substances are substances which modify psychotic behavior. Examples of such agents include phenothiazines, butyrophenones and thioxanthenes.

Anti-emetics are substances which prevent or alleviate nausea or vomiting. An example of such a substance includes dramamine.

Imaging agents are agents capable of imaging a desired site, e.g., tumor, in vivo. Examples of imaging agents include substances having a label which is detectable in vivo, e.g., antibodies attached to fluorescent labels. The term antibody includes whole antibodies or fragments thereof. In certain embodiments, the imaging agent comprises one or more FM® lipophilic styryl dyes such as FM® 1-43, FM® 1-43×, FM® 2-10, FM® 4-64, FM® 4-64× and FM® 5-95, such as FM® 1-43.

In certain embodiments, the payload of the conjugate is a particle. In certain embodiments, the particle is a sensor. In certain embodiments, the particle is a sensor for detecting a cellular analyte. In certain embodiments, the analyte is selected from an ion, e.g., Na⁺, K⁺, Cl⁻, or small molecule, e.g., glucose. In certain embodiments, the sensor can be used to monitor the effects of pharmaceutical agents on biological systems such as the cardiovascular system or the circulatory system. In an exemplary embodiment, the sensor is used to monitor action potentials generated by cardiac or neural cells in culture are defined by a flux of sodium and potassium into and out of the cell. In certain such embodiments, the sensors of the invention measure this ion flux in cardiac cells accurately and spatially in a high throughput manner. In certain other embodiments, the sensors can be used to monitor the fluctuations in glucose levels within cells in response to the intake of food or insulin.

In certain aspects, the sensor particle is for use in the drug discovery process. In certain such embodiments, the sensors are used to measure the efficacy of a therapy. For example, sensors may be employed to monitor the effect of ion channel-modulating drugs. In alternative embodiments, sensors are used to screen for cytotoxic substances by, for example, determining ionic flux in cardiac cells in response to a cytotoxic agent and using these values as a comparison for testing novel therapeutic agents.

In certain aspects, the conjugates are implanted into small animals to monitor biological responses to new therapeutic agents. In certain embodiments, the implantable conjugates are used to study the mechanism of disease in small animals. In certain such embodiments, the animals, such as rats or mice, are, for example, infected with a disease and the biological functions are monitored by detecting the signal of the implanted sensor.

### Exemplification

### Surface Modification

Polystyrene beads (˜0.2 μm from Invitrogen/Molecular Probes or ˜6 μm from Polysciences) with amine functionalities were soaked in a 0.1 M carbonate-bicarbonate buffer overnight. The beads where then pelleted through centrifugation and the buffer was removed. The beads were resuspended an washed in 10 mM Hepes buffer solution. When the 6 μm beads are exposed to an HCl solution, there is visible evolution of bubbles.

In Vitro Testing of Amine Modified Beads

HEK cells were cultured in glass bottom culture dishes with 2 mL of Dulbecco's Modified Eagle Media (10% Fetal Bovine Serum, 1% Antibiotics) and allowed to incubate (37° C., 5% CO₂) for approximately 3.5 hours. The polystyrene beads (˜0.2 μm, Invitrogen) and FM® 1-43 dye (Invitrogen/Molecular Probes) were each warmed to 37° C. prior to loading them into HEK cells. Polystyrene beads (0.5 mL) and a solution of FM 1-43 in dimethylsulfoxide (8 μL of a 1 mM solution, Sigma-Aldrich) were loaded into the culture dishes according to the experimental set-up illustrated in FIG. 5. The cells were then incubated for approximately 19 hours. After incubation, the media was removed and the cells were washed and then resuspended in phosphate buffered saline (37° C., pH=7.4) for imaging. A 510 Meta Zeiss LSM was used to image cells on a 63× oil objective at the wavelengths (excitation/emission) 488/505-545 nm for the polystyrene beads and 488/650-710 for FM 1-43.

1. A conjugate, comprising a payload and an endosome-disrupting component comprising a plurality of moieties that react under acidic conditions to release gaseous endosome-disrupting molecules.

2. The conjugate of claim 1, wherein one or more of the moieties are ionic.

3. The conjugate of claim 1, wherein one or more of the moieties are covalently associated with the conjugate.

4. The conjugate of claim 1, wherein one or more of the moieties are non-covalently associated with the conjugate.

5. The conjugate of claim 1, wherein one or more of the moieties are carbonate ions.

6. The conjugate of claim 1, wherein the endosome-disrupting component comprises a polymer.

7. The conjugate of claim 1, wherein the endosome-disrupting component comprises a dendrimer.

8. The conjugate of claim 1, wherein the endosome-disrupting component comprises a polymeric capsule comprising the moieties.

9. The conjugate of claim 1, wherein the payload is selected from a molecule or particle.

10. The conjugate of claim 9, wherein the payload is a molecule and the molecule is a therapeutic small molecule.

11-12. (canceled)

13. The conjugate of claim 9, wherein the payload is a particle and the particle is a sensor for detecting an analyte.

14-16. (canceled)

17. The conjugate of claim 1, wherein the endosome-disrupting component is associated with the payload through one or more covalent bonds.

18. The conjugate of claim 1, wherein the endosome-disrupting component is associated with the payload through one or more ionic bonds.

19. The conjugate of claim 1, wherein the endosome-disrupting component and the payload are within a biodegradable and/or swellable polymer.

20. The conjugate of claim 19, wherein the biodegradable and/or swellable polymers release the moieties under acidic conditions.

21. The conjugate of claim 1, wherein the endosome-disrupting component reacts under physiologic conditions to release a gas.

22. The conjugate of claim 21, wherein the endosome-disrupting component reacts under physiologic conditions to release CO₂ and/or O₂ molecules.

23-24. (canceled)

25. The conjugate of claim 1, wherein the moieties are selected from carbonates, carbamates, β-keto acids, β-keto esters or any combination thereof.

26. The conjugate of claim 1, wherein one or more of the moieties are metal carbonates.

27. The conjugate of claim 26, wherein the metal carbonates are selected from sodium carbonate, sodium bicarbonate, calcium carbonate, magnesium carbonate, potassium carbonate, potassium bicarbonate, lithium carbonate, and cesium carbonate.

28. The conjugate of claim 1, wherein one or more of the moieties are peroxides.

29. The conjugate of claim 1, wherein the acidic conditions comprise a pH from 4-7.

30-32. (canceled)

33. A method for delivering a payload to a cell, comprising contacting the cell with the conjugate of claim 1.
